(19) United States
(12) Patent Application Publication
(10) Pub. No.: US 2008/0255632 A1
(43) Pub. Date: Oct. 16, 2008

(54) Modulation of the Pain Circuitry to Affect Chronic Pain

(75) Inventor: Ali R. Rezai, Shaker Heights, OH (US)

Correspondence Address: KENYON & KENYON LLP, 1500 K STREET N.W., SUITE 700, WASHINGTON, DC, 20005, US

(73) Assignee: THE CLEVELAND CLINIC FOUNDATION, Cleveland, OH (US)

(21) Appl. No.: 11/938,662

(22) Filed: Nov. 12, 2007

Related U.S. Application Data
(63) Continuation-in-part of application No. 10/502,349, filed on Jul. 23, 2004.

(51) Int. Cl. A61N 1/04

(52) U.S. Cl. 607/46

(57) ABSTRACT

The present invention relates to methods of affecting chronic pain by applying an electrical and/or chemical signal to the target site of the pain circuitry associated with chronic pain. Such target sites include cerebral and deep brain target sites.

### CROSS-REFERENCE TO RELATED APPLICATIONS

The present application is a continuation-in-part of U.S. application Ser. No. 10/502,349, filed on Jan. 31, 2003, which claims the benefit of Provisional U.S. Application No. 60/353,697, filed Feb. 1, 2002, both of which are incorporated by reference herein.

### BACKGROUND OF THE INVENTION

Chronic pain afflicts approximately 86 million Americans and it is estimated that United States business and industry loses about $90 billion dollars annually to sick time, reduced productivity, and direct medical and other benefit costs due to chronic pain among employees. Because of the staggering number of people affected by chronic pain, a number of therapies have been developed that attempt to alleviate the symptoms of this condition. Such therapies include narcotics, non-narcotics, analgesics, antidepressants, anticonvulsants, physical therapy, biofeedback, transcutaneous electrical nerve stimulation (TENS), as well as less conventional or alternative therapies. Other treatment options involve neuroaugmentive techniques such as spinal cord stimulation or intrathecal pumps. For a subset of patients, however, these therapies are inefficacious and more invasive procedures such as blocks, neurolysis and ablative procedures become the only options for treatment. In particular, ablative procedures, although infrequently utilized, are the primary alternative for patients unresponsive to other modes of treatment. Such procedures, however, have the fundamental limitation of being inherently irreversible and being essentially a “one-shot” procedure with little chance of alleviating or preventing potential side effects. In addition, there is a limited possibility to provide continuous benefits as the pathophysiology underlying the chronic pain progresses and the patient's symptoms evolve. Because of the inherent disadvantages of ablative procedures, electrical stimulation of the brain has become an attractive neurosurgical alternative to alleviate the symptoms of chronic pain.

Electrical stimulation of the brain for chronic pain has been used since the 1950s when temporary electrodes were implanted in the septal region for psychosurgery in patients with schizophrenia and metastatic carcinoma. In particular, electrodes were placed in the septum pellucidum in a region anterior and inferior to the foramen of Monro. In the 1960s, there were reports of stimulation of both the caudate nucleus and the septal region in six patients with intractable pain, but successful pain relief was obtained in only one patient. Despite these earlier reports of septal and caudate stimulation, current applications of electrical stimulation for pain involve lateral thalamic, medial lemniscus, internal capsule stimulation, periventricular gray and periaqueductal gray stimulation. For example, thalamic stimulation for pain relief was first reported for stimulation along the ventroposterolateral nucleus and ventralis posterior to relieve chronic intractable deafferentation pain and stimulation along the ventroposteromedial nucleus to relieve refractory facial pain. With respect to internal capsule stimulation, chronic stimulating electrodes have been implanted in the posterior limb of the internal capsule in a number of patients, including patients with lower-extremity pain and spasticity following spinal cord injury.

Although the above-mentioned target sites are all deep brain stimulation target sites, several studies have supported the role of motor cortex stimulation for pain control. For example, in the process of performing sensory cortex stimulation in an attempt to relieve thalamic pain, it was found that stimulation of the precentral gyrus/motor cortex was effective in relieving thalamic pain. Interestingly, stimulation of the sensory cortex exacerbated the pain in many patients.

Therefore, despite previous attempts to alleviate the symptoms of chronic pain by deep brain or cortical stimulation, there is still an unmet need for a method of treating chronic pain that is effective in a larger subset of the patient population.

### SUMMARY OF THE INVENTION

The present invention relates to a method of affecting chronic pain by applying an electrical and/or chemical modulation signal to a target site of the pain circuitry involved in chronic pain. In particular, one embodiment of the present invention provides a method of affecting chronic pain in a patient including positioning a device in a target site of the brain and activating the device to apply a modulation signal to the target site to affect chronic pain. The modulation signal can be an electrical signal and/or a chemical signal. In this embodiment, the target site is selected from the group consisting of the pre-frontal cortex, orbitofrontal cortex, anterior limb of the internal capsule, insular cortex, primary somatosensory cortex, secondary somatosensory cortex, cingulate cortex, anterior cingulate cortex, and posterior cingulate cortex, inferior frontal gyrus, middle frontal gyrus, superior frontal gyrus, medial frontal gyrus, parahippocampal gyrus, precuneus, amygdala, and hippocampus.

Another embodiment of the present invention provides a method of affecting chronic pain in a patient including positioning a device in a target site of the brain and activating the device to provide a modulation signal to the target site. The modulation signal can be an electrical signal and/or a chemical signal. In this embodiment, the target site is selected from the group consisting of the anterior nucleus of the thalamus, intralaminar thalamic nuclei, dorsomedial nucleus of the thalamus, mamillary body, lateral hypothalamus, locus coeruleus, dorsal raphe nucleus, substantia nigra pars compacta, substantia nigral pars reticulata, superior colliculus, tegmentum, ventral tegmentum, tectum, and medial thalamus, nucleus accumbens, ventral striatum, and ventral palladium.

Another embodiment of the present invention provides a method of affecting chronic pain in a patient including positioning a device in a target site of the brain and activating the device to provide a modulation signal to the target site to affect chronic pain. The modulation signal can be an electrical signal and/or a chemical signal. The target site is selected from the group consisting of the periventricular gray, nucleus centerolateralis, periaqueductal gray, Centre Median-Parafascicular (Cm—Pf) complex of the thalamus, ventral striatum, ventral palladium, nucleus accumbens, caudate nucleus, anterior commissure, anterior formix, posterior-medial hypothalamus, subgeniculate area (area 25), putamen, superior parietal lobule, inferior thalamic peduncule, Meynert's nucleus (NBM), ventral anterior globus pallidus, ventral anterior subthalamic nucleus, anterior limb of internal capsule and peri-anterior commissural region, ventral tegmentum, superior colliculus, pre-frontal cortex, orbital frontal cortex, cingulate cortex, amygdala, hippocampus, mammilary bodies, lateral hypothalamus, locus ceruleus, dorsal raphe nucleus, substantia nigra pars compacta, substantia nigra pars reticulata, anterior nucleus of the thalamus, dorsomedial nucleus of the thalamus, superior frontal gyrus, middle frontal gyrus, inferior frontal gyrus, medial frontal gyrus, pre-cuneous, anterior cingulate, post-cingulate, parahippocampal gyrus, and anterior medial ventral palladium.

Another embodiment of the present invention provides a method of affecting chronic pain including positioning a device in communication with a pain circuitry target site and activating the device to provide a modulation signal to modulate the synthesis or release of an endogenous opioid to affect chronic pain.

### BRIEF DESCRIPTION OF THE FIGURES AND TABLES

FIG. 1 is a cross-sectional view of the brain showing placement of a device to practice a method according to the present invention.

Table I provides target sites in the brain for affecting chronic pain and exemplary corresponding stereotactic coordinates for these target sites.

### DETAILED DESCRIPTION OF THE INVENTION

The present invention relates to methods of affecting chronic pain to regulate, prevent, treat, alleviate the symptoms of and/or reduce the effects of chronic pain. Although not wishing to be bound to any particular definition or characterization, chronic pain can generally be characterized as being nociceptive or non-nociceptive pain. Nociceptive pain, also referred to as somatic pain, involves direct activation of the nociceptors, such as mechanical, chemical, and thermal receptors, found in various tissues, such as bone, muscle, vessels, viscera, and cutaneous and connective tissue. The afferent somatosensory pathways are thought to be intact in nociceptive pain and examples of such pain include cancer pain from bone or tissue invasion, non-cancer pain secondary to degenerative bone and joint disease or osteoarthritis, and failed back surgery. The foregoing examples of nociceptive pain are in no way limiting and the methods of the present invention encompass methods of affecting all types of nociceptive pain.

Non-nociceptive pain, also referred to as neuropathic pain, or deafferentation pain, occurs in the absence of activation of peripheral nociceptors. Non-nociceptive pain often results from injury or dysfunction of the central or peripheral nervous system. Such damage may occur anywhere along the neuroaxis and includes thalamic injury or syndromes (also referred to as central pain, supraspinal central pain, or post-stroke pain); stroke; traumatic or iatrogenic trigeminal (trigeminal neuropathic) brain or spinal cord injuries; phantom limb or stump pain; postherpetic neuralgia; anesthesia dolorosa; brachial plexus avulsion; complex regional pain syndrome I and II; postcordotomy dysesthesia; and various peripheral neuropathies. The foregoing examples of non-nociceptive pain are in no way limiting and the methods of the present invention encompass methods of affecting all types of non-nociceptive pain.

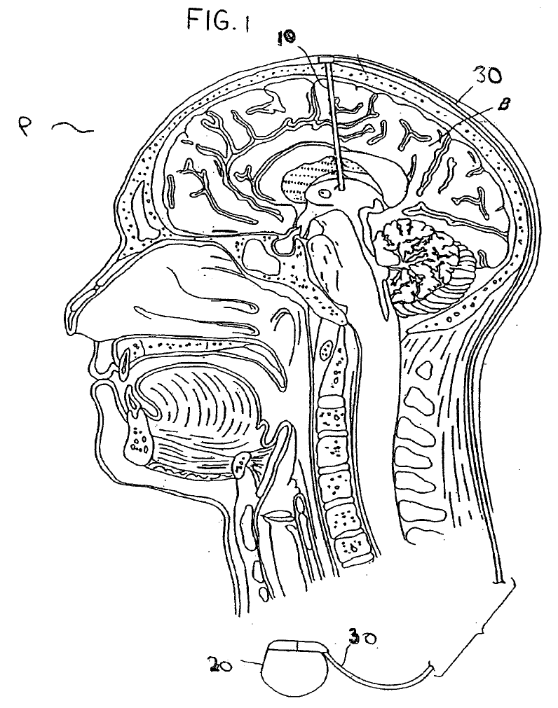

In general, the present invention provides for a method of affecting chronic pain by positioning a device in a pain circuitry target site of the brain. The device can be inserted and/or implanted in the pain circuitry target site. Referring to FIG. 1, in one example of a preferred mode of carrying out a method of the present invention, a device 10, which can be either a catheter or electrode assembly, is implanted within a pain circuitry target site of brain B of a patient P. Device 10 is, in turn, coupled to a device controller 20, which is a pulse generator or drug pump, that generates electrical or chemical modulation signals that are sent to device 10 which are then applied to the pain circuitry target site. For example, the modulation signals can either inhibit or activate neuronal signals in the target site or neuronal signals in a site in communication with the target site. A connector 30, which is an insulated conductor in the case of electrical modulation and an extension of a catheter in the case of chemical modulation, couples modulation controller 20 to device 10. Modulation controller 20 is, in turn, implanted in the chest, abdomen or any other part of a patient P's body and is preferably in patient P's control or is a radio frequency controlled device operated by an external transmitter. In the case of a chemical delivery system where device 10 is a catheter, modulation controller 20 is preferably accessed subcutaneously such that a hypodermic needle can be inserted through the skin to inject a quantity of a chemical agent, such as a neuromodulation agent. The chemical agent is delivered from the modulation controller 20 through a catheter port into the device 10. Modulation controller 20 may be a permanently implanted in patient P or only temporarily implanted such as the temporary neuromodulator described in co-pending U.S. Provisional No. 60/358,176. As stated above, the neuromodulation can be electrical (including ablation) and/or chemical modulation.

The methods of the present invention generally include positioning a device in a pain circuitry target site and activating the device to provide a modulation signal to the pain circuitry target site. In other embodiments, the device is not positioned in the brain but is positioned at another location to modulate a pain circuitry target site. For example, the device can be positioned epidurally, subdurally, or intraparenchymally. By “pain circuitry target site” is meant either a cerebral target site, a deep brain target site or other sites in the brain, as described herein. Table I provides a range of exemplary x, y, and z coordinates of these target sites. Targets are with respect to the Schaltenbrand and Warren Atlas anterior commissure (AC) and posterior commissure (PC) based medial lateral (X), anterior-posterior (Y), and superior-inferior or dorsal-ventral (Z) coordinates with respect to the AC-PC line. As will be readily appreciated by one of skill in the art, targeting of the insular, primary and secondary somatosensory cortex can be achieved by standard neuronavigational techniques which identify standard surface landmarks on the brain. It will also be appreciated that these ranges of coordinates are only exemplary and serve as a general guide to locate these target sites. The coordinates may vary from patient to patient from 2-10 mm, for example. The coordinates may vary more or less than 2-10 mm. However, one skilled in the art can readily locate these sites from patient to patient based on the exemplary coordinates provided in Table I. Targets can also be determined via direct visualization of the structure of imaging such as various MRI sequences, high resolution CT scans, and functional brain imaging methods such as fMRI, MEG and PET. Targets can also be determined according to the Paxinos and Mai atlas of the human brain as well as the Jeanmonod and Morel Atlas of the human thalamus.

TABLE I

| Target Structure | X | Y | Z |
|---|---|---|---|
| Periventricular gray | 1-4 mm lateral to | 2-5 mm anterior to | at AC-PC |
| | wall of 3rd Vent. | PC | |
| Nucleus | 5-9 mm lateral to | 1 mm posterior to | 1 mm below PC to |
| Centerolateralis | wall of 3rd Vent. | PC to 2 mm anterior | 2 mm above PC |
| Periaqueductal gray | 2-4 mm lateral to | At PC up to 4 mm | 2-4 mm below AC- |
| | midline | posterior | PC |
| CM-PF | 6-10 lateral to | 6-10 mm posterior | at AC-PC |
| | midline | to MCP | |
| Ventral Striatum | 5-9 mm lateral to | 0-2 mm anterior to | 2-6 below the AC- |
| | midline | AC | PC |
| Ventral pallidum | 15-30 mm lateral to | 0-6 mm anterior to | 3-10 mm above AC- |
| | midline | MCP | PC |
| Nucleus Accumbens | 5-9 mm lateral to | 0-2 mm anterior to | 2-6 below the AC- |
| | midline | AC | PC |
| Caudate nucleus | 12-25 lateral to | 0-10 mm anterior to | 0-15 mm above AC- |
| | midline | AC | PC |
| Anterior commissure | 0-4 mm lateral to | At AC (12 mm | At AC-PC |
| | midline | anterior to MCP) | |
| Anterior fornix | 1-3 mm lateral to | 0-3 mm posterior to | 0-5 mm superior to |
| | midline | AC | AC-PC |
| Posterior-medial | 1-4 lateral to | 3-5 mm posterior to | 3-5 inferior to AC- |
| hypothalamus | midline | AC | PC |
| Subgeniculate area | 1-6 lateral to | 23-33 mm anterior | 0-5 mm inferior to |
| (area 25) | midline | to AC | AC-PC |
| Putamen | 20-30 mm lateral to | 10 mm posterior to | 5 mm inferior to 15 |
| | midline | 15 mm anterior to | mm superior to AC- |
| | | MCP | PC |
| Superior parietal | On the lateral convexity of the hemispheres. Defined as the area | | |
| lobule | posterior to the central sulcus, lateral to the midline and superior to | | |
| | the intraparietal sucus. | | |
| Inferior thalamic | 5-10 mm lateral | 1-5 mm posterior | 1-4 mm inferior to |
| peduncule | | to AC | AC-PC |
| Meynert's nucleus | 6-15 mm lateral to | 2 mm posterior to | 5-9 mm inferior to |
| (NBM) | midline | 3 mm anterior to AC | AC-PC |
| Ventral anterior | 16-21 mm lateral to | 2-6 mm anterior to | 1-6 mm inferior AC- |
| globus pallidus | midline | MCP | PC |
| Ventral anterior | 10-13 lateral to | 0-3 mm posterior to | 2-6 mm inferior to |
| subthalamic nucleus | midline | MCP | AC-PC |
| Anterior limb of | 7-15 mm lateral to | 3 mm posterior to | 0-15 mm superior to |
| Internal capsule and | midline | 10 mm anterior to | AC-PC |
| peri-anterior | | AC | |
| commissural region | | | |
| Ventral tegmentum | 5-10 mm lateral to | 3-10 mm posterior | 2-5 mm inferior to |
| | midline | to MCP | AC-PC |
| Superior colliculus | 1-10 mm lateral to | 5-10 mm posteror | 0-7 mm inferior to |
| | midline | to PC | AC-PC |
| Pre-frontal cortex | Falx to sphenoid | From 20 mm | From the midline to |
| | ridge | anterior to the | the sylvian fissure |
| | | coronal suture to | |
| | | the base of the skull | |
| Oribital frontal cortex | From the gyrus | From the anterior | Frontal fossa base |
| | rectus to the inferior | commissure to the | to the cingulate |
| | frontal sulcus | frontal pole | sulcus |
| Cingulate cortex | 5-9 mm lateral to | 15-25 mm posterior | 1-5 mm above |
| | midline | to frontal horn tip | ventricular roof |
| Amygdala | 12-22 mm lateral to | 3-15 mm anterior to | 15 to 25 mm inferior |
| | midline | MCP | to AC-PC |
| Hippocampus | Lateral to the | From the amigdala | 10-20 mm inferior to |
| | coronal fissure in | to 40 mm posterior | AC-PC |
| | the floor of the | | |
| | inferior horn | | |
| Mammilary bodies | 0-5 mm lateral to | 2-12 mm anterior to | 5 to 15 mm below |
| | midline | MCP | AC-PC |
| Lateral hypothalamus | 5-15 mm lateral to | 7 mm anterior to 3 | 0-10 mm inferior |
| | midline | mm posterior to AC | below AC-PC |
| Locus ceruleus | 0-7 mm lateral to | 10-20 mm posterior | 5 to 20 mm inferior |
| | midline | to MCP | to AC-PC |
| Dorsal Raphe | 0-7 mm lateral to | 10-20 mm posterior | 3-15 mm inferior to |
| Nucleus | midline | to MCP | AC-PC |
| Substancia Nigra pars | 5-12 mm lateral to | 5 mm anterior to 10 | 5 to 20 mm inferior |
| compacta | midline | mm posterior to | to AC-PC |
| | | MCP | |
| Substancia Nigra pars | 6-15 mm lateral to | 5 mm anterior to 10 | 5 to 20 mm inferior |
| reticulata | midline | mm posterior to | to AC-PC |
| | | MCP | |
| Anterior Nucleus of | 2-12 mm lateral to | 0-10 mm anterior to | 7-15 mm superior to |
| the thalamus | midline | MCP | AC-PC |
| Dorsomedial nucleus | 0-10 mm lateral to | 5 mm anterior to 5 | 5-15 mm superior to |
| of the thalamus | midline | mm posterior to | AC-PC |
| | | MCP | |
| Superior frontal gyrus | 0-26 mm lateral to | 75 mm anterior to | 36-67 mm superior |
| | midline | 19 mm posterior to | to AC-PC |
| | | MCP | |
| Middle frontal gyrus | 25-49 mm lateral to | 72 mm anterior to | 31-67 mm superior |
| | midline | 17 mm posterior to | to AC-PC |
| | | MCP | |
| Inferior frontal gyrus | 33-59 mm lateral to | 7.5-55 mm anterior | 5-31 mm superior to |
| | midline | to MCP | AC-PC |
| Medial frontal gyrus | 0-19.5 mm lateral to | 22-78 mm anterior | 10 mm inferior to 10 |
| | midline | to MCP | mm superior to AC- |
| | | | PC |
| Pre-Cuneous | 0-18 mm lateral to | 37-69 mm posterior | 7.6-63 mm superior |
| | midline | to MCP | to AC-PC |
| Anterior cingulate | 7-10 mm lateral to | 35 mm ant to MCP | 25 mm above AC- |
| | midline | or 2 mm ant to | PC or 7 mm above |
| | | frontal horn | CC |
| Post-cingulate | 0-14 mm lateral to | 0 to 37 mm | 22-36 mm above |
| | midline | posterior to MCP | AC-PC |
| Parahippocampal | 16-29 mm lateral to | 7.67 anterior to 29 | 6-27 mm below AC- |
| gyrus | midline | mm posterior to | PC |
| | | MCP | |
| Anterior Medial | 7-15 mm lateral to | 8 mm anterior to 2 | 0-5 mm below AC- |
| Ventral Pallidum | midline | mm posterior to | PC |
| | | MCP | |
| Anterior Intralaminar | 7-13 mm | MCP to 10 anterior | 0-13 mm |
| Nuclei | | | |
| Posterior Intralaminar | 5-10 mm | MCP: −5 to PC: −7 | 0-13 mm |
| Nuclei | | | |
| Midline Intralaminar | 2-8 mm | MCP to 10 anterior | 0-13 mm |
| Nuclei | | | |
| Medial Thalamus | 0-26 mm | −19 to 75 mm | 36-67 mm |

Therefore, in one embodiment, the present invention provides a method of affecting chronic pain by positioning a device in a cerebral target site and activating the device to provide a modulation signal to the cerebral target site to affect chronic pain. Cerebral target sites according to the present invention include the pre-frontal cortex, cingulate cortex, orbitofrontal cortex, anterior cingulate cortex, posterior cingulate cortex, insular cortex, primary somatosensory cortex, secondary somatosensory cortex, inferior frontal gyrus, middle frontal gyrus, superior frontal gyrus, medial frontal gyrus, parahippocampal gyrus, precuneus, amygdala, and hippocampus. Although the present invention contemplates applying a modulation signal to any one or any combination of cerebral target sites, the particular cerebral target sites can be chosen as a function of the particular effect desired to be achieved. For example, without wishing to be bound by theory, if the emotional, suffering, and motivational components of a patient's chronic pain are desired to be alleviated, then a modulation signal can be applied to the limbic structures including the hippocampus, parahippocampal gyrus, cingulate cortex, and/or the amygdala. If the sensory or discriminatory aspects of pain relay are desired to be alleviated then a modulation signal can be applied to the primary somatosensory cortex, secondary somatosensory cortex, and/or the insular cortex.

In another embodiment, the present invention provides a method of affecting chronic pain by positioning a device in a deep brain target site and providing activating the device to apply a modulation signal to the deep brain target site to affect chronic pain. Deep brain target sites according to the present invention include the anterior nucleus of the thalamus, intralaminar thalamic nuclei, dorsal raphe nucleus, dorsomedial nucleus of the thalamus, locus coeruleus, mammillary bodies, lateral hypothalamus, substantia nigra pars compacta, substantia nigra pars reticulata, superior colliculus, tegmentum, ventral tegmentum, tectum, medial thalamus, nucleus accumbens, ventral striatum, and ventral palladium. Preferred intralaminar thalamic nuclei according to the present invention include the anterior, posterior, and midline intralaminar nuclei. Preferred anterior intrathalamic nuclei include the central lateral, paracentralis, and paralamellar nuclei. Preferred posterior intralaminar nuclei include the centromedian and parafasicularis nuclei. Preferred midline intralaminar nuclei include the paraventricularis and central medial nuclei.

Similar to the method of the present invention directed to applying a modulation signal to cerebral target sites, the present invention contemplates applying a modulation signal to any one or any combination of deep brain target sites. However, particular deep brain target sites can be chosen as a function of the particular effect desired to be achieved. For example, without wishing to be bound by theory, if the emotional, suffering, and motivational components of a patient's chronic pain are desired to be alleviated, then a modulation signal can be applied to the limbic structures, for example, such as the locus coeruleus, lateral hypothalamus, mammillary bodies, and/or anterior thalamic nuclei. If the affective aspects of pain relay are desired to be alleviated then a modulation signal can be applied to the intralaminar thalamic nuclei.

In another embodiment, the present invention provides a method of affecting chronic pain by positioning a device in a target site and activating the device to provide a modulation signal to the target site to affect chronic pain. Table I provides such other target sites such as the periventricular gray, nucleus centerolateralis, periaqueductal gray, centre median-parfascicular complex of the thalamus, caudate nucleus, anterior commissure, anterior formix, posterior-medial hypothalamus, subgeniculate area, putamen, superior parietal lobule, inferior thalamic peduncle, Meynert's nucleus, ventral anterior globus pallidus, ventral anterior subthalamic nucleus, peri-anterior commissural region, and anterior medial ventral palladium.

As stated above, although the stereotactic coordinates for the aforementioned pain circuitry target sites have been provided, the exact location of the target site may vary from patient to patient. Accordingly, standard neurological procedures can be used to localize the x, y, and z coordinates of the target site in a specific patient. For example, a CT scan, an MRI scan, and computerized standard brain atlas can be used to create a 3-dimensional image of a patient's brain and within that image the x, y, and z, coordinates can be identified. Targets can also be determined by direction visualization of the structure as imaged by MRI, high resolution CT, and functional brain imaging such as fMRI, MEG, and PET.

In another embodiment of the present invention, a method of affecting chronic pain includes positioning a device in communication with a pain circuitry target site and providing a activating the device to apply a modulation signal to modulate the synthesis or release of an endogenous opioid to affect chronic pain. Non-limiting examples of endogenous opioids include beta endorphin and metenkephalin. In a preferred embodiment, the pain circuitry target site is the locus coeruleus or the intralaminar thalamic nuclei, including the centromedian, parafasicularis, and the central lateral nuclei. Although not wishing to be bound by theory, by positioning a device in communication with a pain circuitry target site to modulate the synthesis or release of an endogenous opioid, it is intended to modulate the endogenous analgesia pathway, which is thought to include the periaqueductal gray, the nucleus raphe magnus, the locus coeruleus, and the magnocellular part of the nucleus reticularis gigantocellularis. These pathways are also thought to involve descending projections from the midbrain to the dorsal horn as well as various intralaminar nuclei and medial nuclei.

Although this embodiment of the present invention contemplates electrical and/or chemical modulation to modulate the synthesis or release of an endogenous opioid to affect chronic pain, this embodiment is particularly useful for chemical modulation as chemical agents can be delivered directly to the pain circuitry target site. Such chemical agents include antagonists, agonists, other therapeutic neuromodulation agents and any combinations thereof that bind to the receptors of endogenous opioids to regulate the actions of the receptors. Although such chemical agents are generally administered orally in traditional pharmacotherapies, by directly modulating the target sites in the brain that are modulated by such opioids, low and precise doses of the chemical agents can be administered so as to minimize or avoid the side effects and delayed onset of relief common to traditional pharmacotherapy.

With respect to particular details of chemical modulation according to the present invention, whether employed alone or in combination with electrical modulation, once the device (i.e. a catheter) is secured in place in the pain circuitry target site, the modulation controller (i.e. drug pump) is activated thereby delivering a chemical agent to the target site. The chemical agent may be a neurotransmitter mimic; neuropeptide; hormone; pro-hormone; antagonist, agonist, reuptake inhibitor, or degrading enzyme thereof; peptide; protein; therapeutic agent; amino acid; nucleic acid; stem cell or any combination thereof and may be delivered by a slow release matrix or drug pump. In a preferred embodiments, the chemical agent is an antagonist/agonist of an inhibitory neurotransmitter, such as GABA; an excitatory amino acid, such as adenosine; an excitatory neurotransmitter, such as dopamine or glutamate; and/or a neuropeptide, such as substance P. Examples of therapeutic agents include lidocaine, morphine, gabapentin, clonidine, muscimol, or any agents within similar families thereof and any combination of these therapeutic agents. The chemical agents may also be delivered continuously or intermittently.

With respect to particular details of electrical modulation according to the present invention, once the device (i.e. electrode) is secured in place in the pain circuitry target site, the modulation controller (i.e. pulse generator) is activated thereby applying to the target site an oscillating electrical signal having specified pulsing parameters. The oscillating electrical signal may be applied continuously or intermittently and the pulsing parameters, such as the pulse width, amplitude, frequency, voltage, current, intensity, and/or waveform may be adjusted to achieve affect a desired result. Preferably, the oscillating electrical signal is operated at a voltage between about 0.1 μV to about 20 V. More preferably, the oscillating electrical signal is operated at a voltage between about 1 V to about 15 V. Preferably, the electric signal is operated at a frequency range between about 2 Hz to about 2500 Hz. More preferably, the electric signal is operated at a frequency range between about 2 Hz to about 200 Hz. Preferably, the pulse width of the oscillating electrical signal is between about 10 microseconds to about 1,000 microseconds. More preferably, the pulse width of the oscillating electrical signal is between about 50 microseconds to about 500 microseconds. The waveform may be, for example, biphasic square wave, sine wave, or other electrically safe and feasible combination. Preferably, the application of the oscillating electrical signal is: monopolar when the electrode is monopolar, bipolar when the electrode is bipolar, and multipolar when the electrode is multipolar.

The present invention contemplates applying either a chemical or electrical modulation signal and both an electrical and chemical modulation signal to a pain circuitry target site to affect chronic pain. One non-limiting example of the use of chemical and electrical modulation to affect chronic pain, particularly when such chronic pain is characterized by cellular damage at the pain circuitry target site, involves repopulating the target site with undifferentiated cells or nucleic acids and stimulating the growth of such cells or replication of such nucleic acids by electrical modulation. Such repopulation of cells can be carried out using a cellular or molecular approach. Cellular approaches involve injecting or infusing undifferentiated cells, which are preferably cultured autologous cells, into the target site. Molecular approaches involve injecting or infusing nucleic acids, whether in the form of naked or plasmid DNA, into the target site. Methods of delivering nucleic acids to a cellular target site are well known in the art and generally involve the use of delivery vehicles such as expression vector or liposomes. Non-limiting examples of expression vectors for use in this embodiment of the present invention include bacterial expression vectors and viral expression vectors such as retroviruses, adenoviruses, or adeno-associated viral vectors.

In the case of repopulating the target site with nucleic acid molecules, such molecules are preferably recombinant nucleic acid molecules and can be prepared synthetically or, preferably, from isolated nucleic acid molecules, as is known in the art. A nucleic acid is “isolated” when it is purified away from other cellular constituents, such as, for example, other cellular nucleic acids or proteins by standard techniques known to those of skill in the art. The coding region of the nucleic acid molecule can encode a full length gene product or a fragment thereof or a novel mutated or fusion sequence. The coding sequence can be a sequence endogenous to the target cell, or exogenous to the target cell. The promoter, with which the coding sequence is operably associated, may or may not be one that normally is associated with the coding sequence.

The cellular or genetic material can be delivered simultaneously with the electrical modulation, or the cellular or genetic material can be delivered separately. One particularly advantageous feature of this embodiment of combined chemical and electrical modulation is that the expression of the nucleic acid molecules may be regulated by electrical modulation. Namely, the amplitude, intensity, frequency, duration and other pulsing parameters may be used to selectively control expression of nucleic acid molecules delivered to the target site. Further details of the use of electrical modulation and nucleic acid delivery to repopulate a target site are described in U.S. Pat. No. 6,151,525, which describes the use of electrical current to modify contractile cells to form new contractile tissue and which is incorporated by reference herein.

Another example of electrical and chemical modulation being used together, is the use of electrical modulation to modulate the expression of cellular receptors at the target site.

Notwithstanding whether chemical and/or electrical modulation is employed in the methods of the present invention, the present invention also contemplates the use of a closed-loop feedback mechanism in conjunction with chemical or electrical modulation. In such an embodiment, a modulation signal is applied to a pain circuitry target site in response to a detected bodily activity associated with chronic pain. In particular, this embodiment includes positioning a device in communication with a pain circuitry target site, detecting a bodily activity of the body associated with the pain circuitry target site, and providing a modulation signal to a device in response to the detected bodily activity to affect chronic pain. As with the above-described embodiments, the modulation signal can be an electrical and/or chemical signal. Such bodily activity to be detected is any characteristic or function of the body, and includes, for example, respiratory function, body temperature regulation, blood pressure, metabolic activity, cerebral blood flow, pH levels, vital signs, galvanic skin responses, perspiration, electrocardiogram, electroencephalogram, action potential conduction, chemical production, body movement, and response to external modulation. For example, in a preferred embodiment, a patient's threshold to pain could be measured prior to applying a modulation signal to the pain circuitry target site and then the patient's threshold to pain could be measured during application of the modulation signal through the use of tactile modulation or exposure to noxious stimuli to determine the modulation signal. In addition or alternatively, the patient's threshold to increases or decreases in temperature could be measured during modulation of the pain circuitry target site to determine the modulation signal.

In another embodiment of the present invention, the bodily activity of the body includes an electrical or chemical activity of the body and may be detected by sensors located on or within the body. For example, such activity may be detected by sensors located within or proximal to the target site, distal to the target site but within the nervous system, or by sensors located distal to the target site outside the nervous system. Examples of electrical activity detected by sensors located within or proximal to the target site include sensors that measure neuronal electrical activity, such as the electrical activity characteristic of the signaling stages of neurons (i.e. synaptic potentials, trigger actions, action potentials, and neurotransmitter release) at the target site and by afferent and efferent pathways and sources that project to and from or communicate with the target site. For example, if the target site is an intralaminar thalamic nuclei, then sensors can measure, at any signaling stage, neuronal activity of the intralaminar thalamic nuclei and the medial part of the spinothalamic tract, the spinoreticular formation, and the spinomesencephalic tract. In particular, the sensors may detect the rate and pattern of the neuronal electrical activity to determine the modulation signal to be provided to the device.

Examples of chemical activity detected by sensors located within or proximal to the target site include sensors that measure neuronal activity, such as the modulation of neurotransmitters, hormones, pro-hormones, neuropeptides, peptides, proteins, electrolytes, endogenous opioids, or small molecules by the target site and modulation of these substances by afferent and efferent pathways and sources that project to and from the target site or communicate with the target site.

With respect to detecting electrical or chemical activity of the body by sensors located distal to the target site but still within the nervous system, such sensors could be placed in the brain, the spinal cord, cranial nerves, and/or spinal nerves. Sensors placed in the brain are preferably placed in a layer-wise manner in the direction of increasing proximity to the target site. For example, a sensor could be placed on the scalp (i.e. electroencephalogram), in the subgaleal layer, on the skull, in the dura matter, in the sub dural layer and in the parenchyma (i.e. in the frontal lobe, occipital lobe, parietal lobe, temporal lobe) to achieve increasing specificity of electrical and chemical activity detection. The sensors could measure the same types of chemical and electrical activity as the sensors placed within or proximal to the target site as described above.

With respect to detecting electrical or chemical activity by sensors located distal to the target site outside the nervous system, such sensors may be placed in venous structures and various organs or tissues of other body systems, such as the endocrine system, muscular system, respiratory system, circulatory system, urinary system, integumentary system, and digestive system or such sensors may detect signals from these various body systems. For example, with respect to the respiratory system, sensors could detect lung function such as signs of hyperventilation as a measurement of chronic pain; with respect to the circulatory system, sensors could detect leg discoloration, as a measurement of chronic pain; with respect to the integumentary system, sensors could detect perspiration or response to tactile modulation as a measurement of chronic pain; with respect to the muscular system, sensors, such as accelerometers, could detect physical activity of the body such as head movements. All the above-mentioned sensing systems may be employed together or any combination of less than all sensors may be employed together.

After the sensor(s) detect the relevant bodily activity associated with the pain circuitry target site, the sensors generate a sensor signal. The sensor signal is processed by a sensor signal processor and provides a control signal to the modulation controller, which is a signal generator or drug pump depending on whether electrical or chemical modulation is desired. The modulation controller, in turn, generates a response to the control signal by providing a modulation signal to the device. The device then applies the modulation signal to the target site to affect chronic pain. In the case of electrical modulation, the control signal may be an indication to initiate, terminate, increase, decrease or change the rate or pattern of a pulsing parameter of the electrical modulation and the modulation signal can be the respective initiation, termination, increase, decrease or change in rate or pattern of the respective pulsing parameter. In the case of chemical modulation, the control signal can be an indication to initiate, terminate, increase, decrease or change the rate or pattern of the amount or type of chemical agent administered, and the modulation signal can be the respective initiation, termination, increase, decrease or change in the rate or pattern of the amount or type of chemical agent administered. The processing of closed-loop feedback systems for electrical and chemical modulation are described in more detail in respective U.S. Pat. Nos. 6,058,331 and 5,711,316, both of which are incorporated by reference herein.

Although the application of sensors to detect bodily activity are within the scope and spirit of the present invention, the present invention also contemplates the relevant bodily activity to be detected without sensors. For example, signs of hyperventilation and leg discoloration, as well as visual analogs and pain scores can be made or analyzed by visual observation without the assistance of sensors. In such case the modulation signal could still be an initiation, termination, increase, decrease, or change in the rate or pattern of electrical and/or chemical modulation in response to the visual observation.

Although not wishing to be bound by the description of a particular procedure, one exemplary procedure effectuating the methods of the present invention shall now be described with respect to electrical modulation of a pain circuitry target site. Generally, the procedure begins with the patient having a stereotactic head frame mounted to the patient's skull, although frameless techniques may also be used. The patient then typically undergoes a series of MRI and/or CT sessions, during which a series of two dimensional slice images of the patient's brain are built up into a quasi-three dimensional map in virtual space. This map is then correlated to the three dimensional stereotactic frame of reference in the actual surgical field. In order to align these two coordinate frames, both the instruments and the patient should be situated in correspondence to the virtual map. A current method of achieving this alignment is to rigidly mount to the head frame to the surgical table. Subsequently, a series of reference points are established relative to aspects of the frame and patient's skull, so that a computer can adjust and calculate the correlation between the actual surgical field of the patient's head and the virtual space model of the patient's brain MRI scans. Initial anatomical localization of the pain circuitry target site is achieved either directly using the MRI images, or indirectly using interactive anatomical atlas programs that map the atlas image onto the stereotactic image of the brain. This indirect targeting approach involves entering the stereotactic anterior commissure (AC) and posterior commissure (PC) coordinates into a computer with a commercially available program containing digitized diagrams of sagittal brain sections from a standardized brain atlas. The program transcribes the patient's calculated AC-PC intercommissural line onto the digitized map at the sagittal laterality of interest. On these maps, the pain circuitry targets sites can be localized. The subsequently generated map is overlaid onto a millimeter grid ruled in stereotactic coordinates in the anteroposterior and dorsoventral scales with a corresponding diagram of the brain nuclei and tracts depicted in the chosen laterality. The laterality of the maps is chosen according to the location of the pain. Typical laterality is 12 to 14 millimeters from the midline for facial pain, 14 to 15 mm for upper extremity pain, and 15 to 17 millimeters for lower-extremity pain.

Another method of localizing the pain circuitry target site involves the fusion of functional and structural medical imaging. Such methods for localizing targets in the body and guiding diagnostic or therapeutic instruments toward a target region in the body have been described in U.S. Pat. No. 6,368,331, issued on Apr. 9, 2002 to Front et al., U.S. Patent Application Publication No. US 2002/0032375, published Mar. 14, 2002 by Bauch et al., and U.S. Patent Application Publication No. US 2002/0183607, published Dec. 5, 2002 by Bauch et al., all of which are hereby incorporated by reference in their entireties. Methods for target localization specifically within the nervous system, including the brain, have been described in U.S. Provisional Application No. 60/353,695, filed Feb. 1, 2002, by Rezai et al. which is hereby incorporated by reference in its entirety. Specifically, provided in U.S. Provisional Application No. 60/353,695 is a method of medical imaging, comprising: placing a fiducial marker proximate to an area of a body to be imaged; obtaining a first image of the area of the body using a first medical imaging technique, the first image including a first image of the fiducial marker; obtaining a second image of the area of the body using a second medical imaging technique, the second image including a second image of the fiducial marker, the second medical imaging technique being different than the first medical imaging technique; superimposing the first image of the area of the body and the second image of the area of the body; and aligning the first image of the first fiducial marker with the second image of the fiducial marker. Useful medical imaging techniques to obtain functional images include but are not limited to functional MRI, PET or MEG. Useful medical imaging techniques to obtain structural images include but are not limited to volumetric MRI and CT.

Subsequent to the stereotactic imaging (or functional and structural imaging), acquisition of the images, and anatomical localization, the patient is taken to the operating room. The surgery can be performed under either local or general anesthetic, but preferably under local anesthesia in order to allow communication with the patient. An initial incision is made in the scalp, preferably 2.5 centimeters lateral to the midline of the skull, anterior to the coronal suture. A burr hole is then drilled in the skull itself; the size of the hole being suitable to permit surgical manipulation and implantation of an electrode. This size of the hole is generally about 14 millimeters. The dura is then opened, and fibrin glue is applied to minimize cerebral spinal fluid leaks and the entry of air into the cranial cavity. A guide tube cannula with a blunt tip is then inserted into the brain parenchyma to a point approximately one centimeter from the target tissue. At this time physiological localization starts with the ultimate aim of correlating the anatomical and physiological findings to establish the final stereotactic target structure.

Physiological localization using single-cell microelectrode recording is preferably performed for definitively identifying the pain circuitry target site by neuronal firing patterns of individual neurons. Single-cell microelectrode recordings obtained from intralaminar thalamic cells typically have a characteristic bursting activity. In addition to microelectrode recording, microstimulation and or macrostimulation may be performed to provide further physiological localization.

Once the final pain circuitry target site has been identified in the actual spatial frame of reference, the electrode is inserted into the target site and a hand-held pulse generator (Screener) is used for intraoperative test stimulation. Various pole combinations and modulation frequency, pulse width, and intensity are used to determine the thresholds for therapeutic and adverse effects. Thereafter the electrode is locked into the burr hold ring to prevent lead migration. The proximal portion of the electrode is then attached to a transcutaneous pacing wire for a test trial period. After the test period, the patient undergoes implantation of a pulse generator or radio-frequency-coupled receiver.

Implanting the pulse generator is generally carried out with the patient under general anesthesia. The pulse generator is implanted in the infraclavicular space by tunneling from the frontal incision to the infraclavicular space. The pulse generator can be powered by a battery and can be activated externally by an external transmitter.

Although the invention has been described with reference to the preferred embodiments, it will be apparent to one skilled in the art that variations and modifications are contemplated within the spirit and scope of the invention. The figures, tables, and description of the preferred embodiments are made by way of example rather than to limit the scope of the invention, and it is intended to cover within the spirit and scope of the invention all such changes and modifications. These coordinates are only exemplary and can deviate based on patient's head size. These are standard ranges but are not limiting and are only exemplary and of course are not limiting.

What is claimed is:

1. A method of affecting chronic pain in a patient comprising:
a) positioning a device in a target site of the brain; and
b) activating the device to apply a modulation signal to the target site to affect chronic pain, the target site selected from the group consisting of the periventricular gray, nucleus centerolateralis, periaqueductal gray, Centre Median-Parafascicular (Cm—Pf) complex of the thalamus, ventral striatum, ventral palladium, nucleus accumbens, caudate nucleus, anterior commissure, anterior fornix, posterior-medial hypothalamus, subgeniculate area (area 25), putamen, superior parietal lobule, inferior thalamic peduncule, Meynert's nucleus (NBM), ventral anterior globus pallidus, ventral anterior subthalamic nucleus, anterior limb of internal capsule and peri-anterior commissural region, ventral tegmentum, superior colliculus, pre-frontal cortex, orbital frontal cortex, cingulate cortex, amygdala, hippocampus, mammilary bodies, lateral hypothalamus, locus ceruleus, dorsal raphe nucleus, substantia nigra pars compacta, substantia nigra pars reticulata, anterior nucleus of the thalamus, dorsomedial nucleus of the thalamus, superior frontal gyrus, middle frontal gyrus, inferior frontal gyrus, medial frontal gyrus, pre-cuneous, anterior cingulate, post-cingulate, parahippocampal gyrus, and anterior medial ventral palladium.

2. The method of claim 1, wherein the target site is the periventricular gray.

3. The method of claim 1, wherein the target site is the nucleus centerolateralis.

4. The method of claim 1, wherein the target site is the periaqueductal gray.

5. The method of claim 1, wherein the target site is the Centre Median-Parafascicular (Cm—Pf) complex of the thalamus.

6. The method of claim 1, wherein the target site is the thalamus.

7. The method of claim 1, wherein the target site is the ventral striatum.

8. The method of claim 1, wherein the target site is the ventral palladium.

9. The method of claim 1, wherein the target site is the nucleus accumbens

10. The method of claim 1, wherein the target site is the caudate nucleus.

11. The method of claim 1, wherein the target site is the anterior commissure.

12. The method of claim 1, wherein the target site is the anterior formix.

13. The method of claim 1, wherein the target site is the posterior-medial hypothalamus.

14. The method of claim 1, wherein the target site is the subgeniculate area (area 25

15. The method of claim 1, wherein the target site is the putamen.

16. The method of claim 1, wherein the target site is the superior parietal lobule.

17. The method of claim 1, wherein the target site is the inferior thalamic peduncule.

18. The method of claim 1, wherein the target site is the Meynert's nucleus (NBM).

19. The method of claim 1, wherein the target site is the ventral anterior globus pallidus.

20. The method of claim 1, wherein the target site is the ventral anterior subthalamic nucleus.

21. The method of claim 1, wherein the target site is the anterior limb of internal capsule and peri-anterior commissural region

22. The method of claim 1, wherein the target site is the superior colliculus.

23. The method of claim 1, wherein the target site is the pre-frontal cortex.

24. The method of claim 1, wherein the target site is the orbitofrontal cortex.

25. The method of claim 1, wherein the target site is the cingulate cortex.

26. The method of claim 1, wherein the target site is the amygdala

27. The method of claim 1, wherein the target site is the hippocampus.

28. The method of claim 1, wherein the target site is the mammilary bodies.

29. The method of claim 1, wherein the target site is the lateral hypothalamus.

30. The method of claim 1, wherein the target site is the locus ceruleus.

31. The method of claim 1, wherein the target site is the substantia nigra pars compacta.

32. The method of claim 1, wherein the target site is the substantia nigra pars reticulata.

33. The method of claim 1, wherein the target site is the anterior nucleus of the thalamus.

34. The method of claim 1, wherein the target site is the dorsomedial nucleus of the thalamus.

35. The method of claim 1, wherein the target site is the superior frontal gyrus.

36. The method of claim 1, wherein the target site is the middle frontal gyrus.

37. The method of claim 1, wherein the target site is the inferior frontal gyrus.

38. The method of claim 1, wherein the target site is the medial frontal gyrus.

39. The method of claim 1, wherein the target site is the pre-cuneous

40. The method of claim 1, wherein the target site is the anterior cingulate.

41. The method of claim 1, wherein the target site is the post-cingulate.

42. The method of claim 1, wherein the target site is the parahippocampal gyrus.

43. The method of claim 1, wherein the target site is the and anterior medial ventral palladium.

44. The method of claim 1, wherein the target site is the dorsal raphe nucleus.

45. The method of claim 1, wherein the target site is the ventral tegmentum.

46. The method of claim 1, wherein the modulation signal is an electrical signal, a chemical signal, or both.
